(19) United States
(12) Patent Application Publication
(10) Pub. No.: US 2015/0133738 A1
(43) Pub. Date: May 14, 2015

(54) DEVICE AND METHOD FOR LIFTING ABDOMINAL WALL DURING MEDICAL PROCEDURE

(71) Applicant: Life Care Medical Devices, Ltd., New Smyrna Beach, FL (US)

(72) Inventor: Glenn S. Foley, Santa Rosa, CA (US)

(73) Assignee: Life Care Medical Devices, Ltd.

(21) Appl. No.: 14/598,911

(22) Filed: Jan. 16, 2015

Related U.S. Application Data
(63) Continuation of application No. 13/640,015, filed on Jan. 3, 2013, now abandoned.

(51) Int. Cl. A61B 19/00; A61M 1/00; A61B 17/34; A61B 1/32; A61M 13/00; A61B 17/02; A61M 39/02

(52) U.S. Cl. CPC A61B 19/24; A61B 17/0281; A61B 17/0293; A61M 1/0066; A61M 39/02; A61B 1/32; A61B 17/0218; A61M 13/003; A61B 17/3423; A61B 2017/308
USPC 600/204

(57) ABSTRACT

A device (100) and method (200) for elevating skin; subcutaneous tissue, and muscle of a patient during medical procedure, the device comprises at least one opening (102), at least one at least one ring liner (106). The opening (102) located at the top of the device having means for inserting surgical instruments within the patient's body cavity created by separation of the skin, subcutaneous tissue, and muscle from the underlying organs or anatomical features cavity. The pneumatic suction port (104) is externally connected through tubing with an external vacuum system having means for allowing negative pressure to be introduced into the patient's body cavity and the ring liner (106) having means for a soft interface between the device and the skin and provide a positive seal between the device and the skin.

### FIELD OF INVENTION

The present invention relates to a device and method for elevating skin, subcutaneous tissue and muscle of a patient from the underlying organs or anatomical features cavity to facilitate safe entry of surgical instruments during a medical procedure.

### BACKGROUND ART

Conventional medical procedure requires direct incision on the abdominal wall for insertion of surgical instruments such as trocar into the patient's body cavity. The incision must be sufficiently large to enable the insertion of surgical instruments to be utilized by surgeons as well as to provide a viewing space for medical procedure through insertion of a laparoscope wherein the laparoscope is connected to a video camera with means to project a magnified view of the operative field onto a monitor which facilitates performance of complex medical procedure. Insufflation of the patient's body cavity with inert gas such as carbon dioxide, CO₂ to elevate the abdominal wall from underlying vital organs within the patient's body cavity is for the purpose of creating a working space for the medical procedure within the patient's body cavity. CO₂ is used as it is common to the human body and can be absorbed by tissue and is a waste product of respiration. In addition, it is not readily combusted by flame which is an advantage to medical surgery with the use of electrosurgical devices. The presence of gas within the patient's body cavity is known as pneumoperitoneum.

However, the challenge of the conventional approach for accessing patients' body cavity for medical procedure lies in the lifting of the abdominal wall wherein conventionally, devices such as retractors are used to lift the abdominal wall of the patient's body cavity to enable incision or perforations on the patient's abdominal wall by means of trocars. The use of retractors or lifting rods delivers a lifting force for elevation or separation of the skin, subcutaneous tissue and muscle of a patient from the underlying organs or anatomical features cavity to facilitate safe entry of medical devices for medical procedure.

In general, typical devices for elevation of skin, subcutaneous tissue and muscle of a patient from the underlying organs such as the retractors are predominantly bulky and require complex installation procedure. In addition, the lifting force produced by these bulky devices consumes to risk of vascular injury as well as incision-related complications such as dehiscence and hernia upon due to large incisions.

The other disadvantages of the conventional approach in accessing the abdominal wall of the patient's body cavity lies in the creation of pneumoperitoneum which requires special pumps, pressure gauges and needles and the possibility of gas seeping into the blood. In addition, the insufflation gas periodically seeps out of the abdominal cavity, whereupon the abdominal wall falls and interrupts the surgery.

The device of the present invention which utilizes negative pressure to raise the abdominal wall above the vital organs to facilitate safe entry of surgical instruments may reduce the risk of some complications associated with ‘blind entry’ and reduce the size of the myofascial defect in the abdominal wall. The methodology of the present invention for elevating skin, subcutaneous tissue and muscle of a patient from the underlying organs which utilizes negative pressure allows for CO₂ to be introduced upon sufficient content of negative pressure to maintain the insufflations level of the abdominal cavity for safe entry of surgical instruments for the medical procedure. Thus, the device and methodology of the present invention has numerous advantages over all relevant prior art.

The subject matter claimed herein is not limited to embodiments that solve any disadvantages or that operate only in environments such as those described above. Rather, this background is only provided to illustrate one exemplary technology area where some embodiments described herein can be practiced.

### SUMMARY OF INVENTION

In one embodiment of the present invention is a device for elevating skin, subcutaneous tissue, and muscle of a patient to facilitate safe entry of surgical instruments for medical procedure. The device comprises at least one opening, at least one pneumatic suction port and at least one ring liner. The opening located at the top of the device having means for inserting surgical instruments within the patient's body cavity created by separation of the skin, subcutaneous tissue, and muscle from the underlying organs or anatomical features cavity. The pneumatic suction port is externally connected through tubing with an external vacuum system having means for allowing negative pressure to be introduced into the patient's body cavity and the ring liner having means for a soft interface between the device and the skin and provide a positive seal between the device and the skin.

In another embodiment of the present invention is the method for elevating skin, subcutaneous tissue, and muscle of a patient to facilitate safe entry of surgical instruments during medical procedure. The method comprising the steps of placing a device on the patient's target medical intraventional area for elevating the target medical intraventional area of a patient from underlying organs in the patient's body, exerting negative pressure onto the target body area of the patient through the suction port to create a pneumoperitoneum and inserting surgical instruments into the patient's abdomen for medical procedure through the opening at the top of the device.

The present invention consists of several features and a combination of parts hereinafter fully described and illustrated in the accompanying drawings, it being understood that various changes in the details may be made without departing from the scope of the invention or sacrificing any of the advantages of the present invention.

### BRIEF DESCRIPTION OF ACCOMPANYING DRAWINGS

To further clarify various aspects of some embodiments of the present invention, a more particular description of the invention will be rendered by references to specific embodiments thereof, which are illustrated in the appended drawings. It is appreciated that these drawings depict only typical embodiments of the invention and are therefore not to be considered limiting of its scope. The invention will be described and explained with additional specificity and detail through the accompanying drawings in which:

FIG. 1A illustrates a device for elevating skin, subcutaneous tissue, and muscle of a patient to facilitate safe entry of surgical instruments for medical procedure.

FIG. 1B illustrates a block diagram of a device for elevating skin, subcutaneous tissue, and muscle of a patient to facilitate safe entry of surgical instruments for medical procedure.

FIG. 2 is a flowchart illustrating a method for elevating skin, subcutaneous tissue, and muscle of a patient to facilitate safe entry of surgical instruments for medical procedure.

FIG. 3 is a flowchart illustrating a method for exerting negative pressure onto the target body area of the patient through the suction port to create a pneumoperitoneum.

### DETAILED DESCRIPTION OF THE PREFERRED EMBODIMENTS

Embodiments of the invention relate to a device and a method for elevating skin, subcutaneous tissue, and muscle of a patient to facilitate safe entry of surgical instruments for medical procedure by raising the abdominal wall above the underlying organs or anatomical features cavity through pneumatic means. Hereinafter, this specification will describe the present invention according to the preferred embodiments of the present invention. However, it is to be understood that limiting the description to the preferred embodiments of the invention is merely to facilitate discussion of the present invention and it is envisioned without departing from the scope of the appended claims. The present invention describes a device and a method for elevating skin, subcutaneous tissue, and muscle of a patient to facilitate safe entry of surgical instruments for medical procedure upon establishment of a pneumoperitoneum in the patient's body cavity.

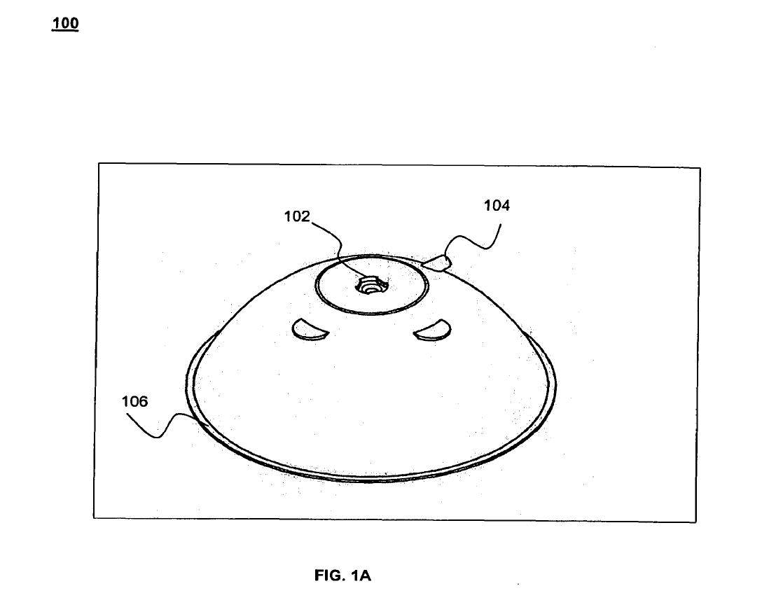

Reference is first being made to FIGS. 1A and 1B collectively. FIG. 1A illustrates a device for elevating skin, subcutaneous tissue, and muscle of a patient to facilitate safe entry of surgical instruments for medical procedure while FIG. 1B illustrates a block diagram of a device for elevating skin, subcutaneous tissue, and muscle of a patient to facilitate safe entry of surgical instruments for medical procedure. The device for elevating skin, subcutaneous tissue, and muscle of a patient to facilitate safe entry of surgical instruments for medical procedure comprises at least one opening (102), at least one pneumatic suction port (104) and at least one ring liner (106). The device (100) is a dome shaped device and is placed on the patient's target medical intraventional area without any fastening means. In addition, the device utilizes negative pressure through pneumatic means to raise the abdominal wall above the underlying organs of the patient's body cavity. The said pneumatic principle is clearly observed in the present invention as negative pressure is introduced through an external vacuum system to the pneumatic suction port (104) of the device wherein the pneumatic suction port (104) is externally connected through tubing to the said vacuum system.

The opening (102) which is located at the top of the device (100) provide means for inserting surgical instruments within the patient's body cavity created by separation of the skin, subcutaneous tissue, and muscle from the underlying organs or anatomical features cavity. The opening (102) further comprises a conduit for insertion of a plurality of surgical instrument which enables the distal ends of the said instruments to project into the patient's body cavity. The said opening (102) further comprises a seal which at least partially seals the opening to inhibit the escape of insufflation gas. A positive seal against atmospheric pressure is provided by the ring liner (106) which lines the bottom of the dome shaped device.

The device (100) of the present invention is made to fit an insufflated abdominal shape and is removable from the patient's abdomen for medical procedure upon replacement of negative pressure with positive pressure inert gas. Thereafter, surgeons may remove the device (100) upon elevation of the skin, subcutaneous tissue, and muscle of a patient of the body cavity and perform the medical procedure on the target medical intraventional area by inserting laparoscopic instruments directly onto the patient's abdomen. However, the surgeon may choose to leave the device (100) on the patient's target medical intraventional area upon elevation of the skin, subcutaneous tissue, and muscle of a patient of the body cavity and insert the required surgical instruments through the opening (102) at the top of the device (100). The said device (100) is disposable device.

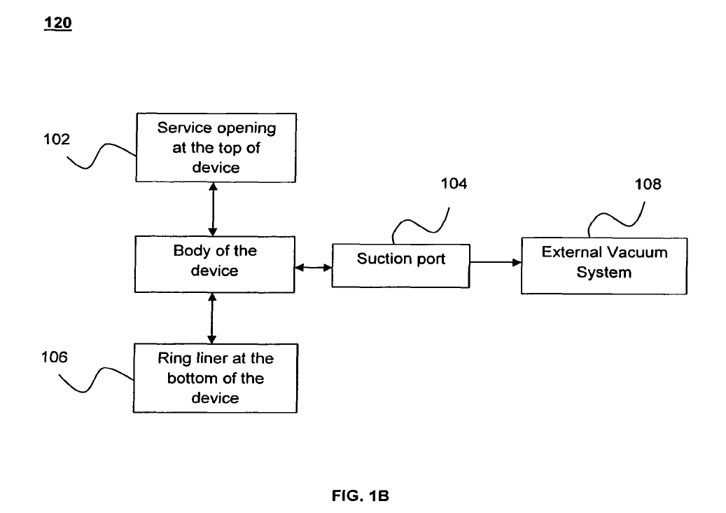

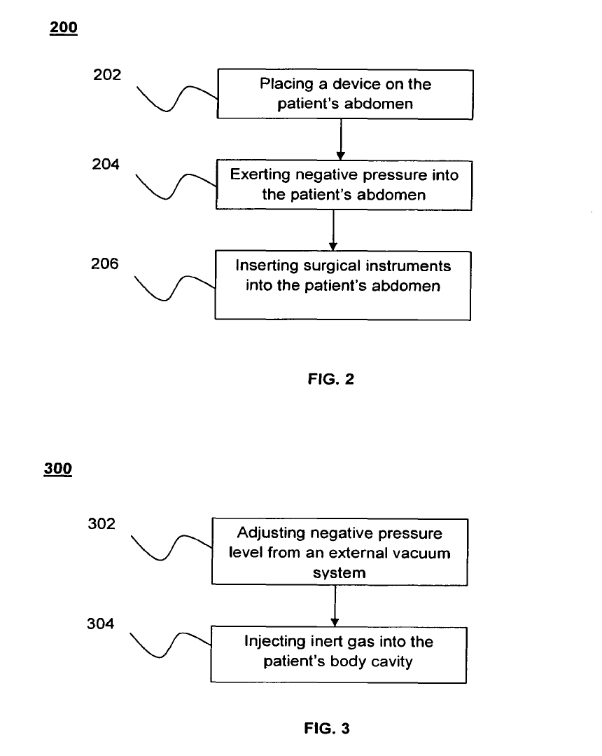

Reference is now being made to FIGS. 2 and 3 collectively. FIG. 2 is a flowchart illustrating a method for elevating skin, subcutaneous tissue, and muscle of a patient to facilitate safe entry of surgical instruments for medical procedure while FIG. 3 is a flowchart illustrating a method for exerting negative pressure onto the target body area of the patient through the suction port to create a pneumoperitoneum. The method (200) for elevating skin, subcutaneous tissue, and muscle of a patient during medical procedure comprising the steps of placing a device on the patient's target medical intraventional area for elevating the target medical intraventional area of a patient from underlying organs in the patient's body (202), exerting negative pressure onto the target body area of the patient through the suction port (104) to create a pneumoperitoneum (204) and inserting surgical instruments into the patient's abdomen for medical procedure through the opening at the top of the device (206).

Negative pressure is introduced into the pneumatic suction port (104) of the device through adjustment of the negative pressure level on the external vacuum system by adjusting the negative pressure level from an external vacuum system which is connected to the suction port to control and ensure sufficient negative pressure is present to elevate the skin, subcutaneous tissue, and muscle of a patient away from underlying organs within the patient's body cavity (302). Thereafter, inert gas such as CO₂ which is common to the human body is introduced into the patient's body cavity through insertion of an instrument through the opening at the top of the device to increase separation between the skin, subcutaneous tissue, and muscle from the underlying organs within the patient's body cavity when negative pressure reaches a sufficient level (304).

Surgical instruments are inserted into the patient's body cavity through the opening located at the top of the device into a conduit of the said opening. Alternatively the said surgical instruments may be inserted directly on the patient's abdomen to enable distal ends of the said instruments to project into the patient's body cavity upon elevation of the skin, subcutaneous tissue, and muscle of a patient from the underlying organs of the patient's body cavity in a pneumoperitoneum.

The device and methodology of the present invention is useful as it prevents collapse of the abdominal wall of the patient's body cavity by introducing sufficient negative pressure and positive pressure inert gas which facilitates safe entry of surgical instruments. Safe entry of surgical instruments onto the patient's body cavity ensures less scaring as well as a less traumatic operation. The device of the present invention as described is intended to a more simple yet versatile and convenient to be used in operating theatres to assist surgeons in medical procedure.

The present invention may be embodied in other forms without departing from its essential characteristics. The described embodiments are to be considered in all respects only as illustrative and not restrictive. The scope of the invention is, therefore indicated by the appended claims rather than by the foregoing description. All changes, which come within the meaning and range of equivalency of the claims, are to be embraced within their scope.

1. A device (100) for elevating skin, subcutaneous tissue, and muscle of a patient during medical procedure, the device comprises:
at least one opening (102);
at least one pneumatic suction port (104); and
at least one ring liner (106)
characterized in that
the opening (102) located at the top of the device having means for inserting surgical instruments within the patient's body cavity created by separation of the skin, subcutaneous tissue, and muscle from the underlying organs or anatomical features cavity;
the pneumatic suction port (104) is externally connected through a tubing with an external vacuum system having means for allowing negative pressure to be introduced into the patient's body cavity; and
the ring liner (106) having means for a soft interface between the device and the skin and provide a positive seal between the device and the skin.

2. The device (100) according to claim 1, is placed on the patient's target medical intraventional area without any fastening means.

3. The device (100) according to claim 1, wherein the opening (102) further comprises a conduit for insertion of a plurality of surgical instruments which enables the distal ends of said instruments to project into the patient's body cavity.

4. The device (100) according to claim 1 wherein the opening (102) further comprises a seal which at least partially seals the opening to inhibit the escape of insufflation gas.

5. The device (100) according to claim 1, is removable from the patient's abdomen for medical procedure upon replacement of negative pressure with positive pressure inert gas.

6. The device (100) according to claim 1, is made to fit an insufflated abdominal shape.

7. The device (100) according to claim 1 is a disposable device.

8. The device (100) according to claim 1, wherein the ring liner (106) provides a positive seal against atmospheric pressure.

9. The use of the device (100) according to claim 1, for elevating skin, subcutaneous tissue, and muscle of a patient during medical procedure.

10. A method (200) for elevating skin, subcutaneous tissue, and muscle of a patient during medical procedure comprising the steps of:
placing a device on the patient's target medical intraventional area for elevating the target medical intraventional area of a patient from underlying organs in the patient's body (202);
exerting negative pressure onto the target body area of the patient through the suction port (104) to create a pneumoperitoneum (204); and
inserting surgical instruments into the patient's abdomen for medical procedure through the opening at the top of the device (206).

11. The method according to claim 10, wherein exerting negative pressure onto the target body area through the suction port to create pneumoperitoneum (204) further comprises the steps of:
adjusting negative pressure level from an external vacuum system connected to the suction port to control and ensure sufficient negative pressure is present to elevate the skin, subcutaneous tissue, and muscle of a patient away from underlying organs within the patient's body cavity (302); and
injecting inert gas into the patient's body cavity through insertion of an instrument through the opening at the top of the device to increase separation between the skin, subcutaneous tissue, and muscle from the underlying organs within the patient's body cavity when negative pressure reaches a sufficient level (304).

12. The method according to claim 10, wherein inserting surgical instruments into the patient's abdomen for medical procedure (206) further comprises inserting at least one surgical instrument through the opening located at the top of the device into a conduit of the said opening or directly on the patient's abdomen to enable distal ends of the said instruments to project into the patient's body cavity.
